(19) United States
(12) Patent Application Publication
(10) Pub. No.: US 2006/0142662 A1
(43) Pub. Date: Jun. 29, 2006

(54) Analysis apparatus and method comprising auto-focusing means

(76) Inventor: Michael Cornelis Van Beek, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/560,453

(22) PCT Filed: Jun. 15, 2004
(86) PCT No.: PCT/IB04/50911

§ 371 Date: Dec. 14, 2005

(30) Foreign Application Priority Data
Jun. 19, 2003 (EP) 03101806.2

(51) Int. Cl. A61B 5/00

(52) U.S. Cl. 600/476

(57) ABSTRACT

The present invention relates to an analysis apparatus, in particular a spectroscopic analysis apparatus, for analyzing an object, such as blood of a patient, and a corresponding analysis method. To aim the confocal detection volume inside a blood vessel orthogonal polarized spectral imaging (OPS imaging) is used to locate blood capillaries in the skin. Image processing means (ipm) determining image characteristics, which indicate if an imaging system (img) for imaging the object is focused on the object (obj) to be analyzed, from a detected image. Said image processing means (ipm) are preferably adapted for determining the amplitudes of spatial frequencies corresponding to typical characteristics of the object (obj) from a detected image, or for determining the maximum contrast present in a detected image. Based on the determined image characteristics, auto-focusing means (afm) control the focusing means (mo) to change the focusing accordingly, whereafter the object is imaged and the same image characteristics are determined again from a the new image. This is preferably repeatedly done until the object (obj) substantially lies in a detection plane (dp) onto which the focusing means (mo) are focused. Thus, continuous autofocusing with high accuracy can be achieved.

The present invention relates to an analysis apparatus, in particular a spectroscopic analysis apparatus, for analyzing an object, such as the blood of a patient, and a corresponding analysis method. Further, the invention relates to an optical focusing system for focusing on a target point of an object.

In general, analysis apparatuses, such as spectroscopic analysis apparatuses, are used to investigate the composition of an object to be examined, e.g. to measure the concentration of various analytes in blood in vivo. In particular, analysis apparatuses employ an analysis, such as a spectroscopic decomposition, based on interaction of the matter of the object with incident electromagnetic radiation, such as visible light, infrared or ultraviolet radiation.

A spectroscopic analysis apparatus comprising an excitation system and a monitoring system is known from WO 02/057759 A2 which is incorporated herein by reference. The excitation system emits an excitation beam to excite a target region during an excitation period. The monitoring system emits a monitoring beam to image the target region during a monitoring period. The excitation period and the monitoring period substantially overlap. Hence the target region is imaged together with the excitation, and an image is formed displaying both the target region and the excitation area. On the basis of this image, the excitation beam can be very accurately aimed at the target region.

The analysis method known from WO 02/057759 A2 for simultaneous imaging and spectral analysis of a local composition is done by separate lasers for confocal video imaging and Raman excitation or by use of a single laser for combined imaging and Raman spectroscopic analysis. Orthogonal polarized spectral imaging (OPS imaging), which is also described in WO 02/057759 A2, is a simple, inexpensive and robust method to visualize blood vessels close to the surface of organs which can also be used to visualize blood capillaries in the human skin. Blood capillaries close to the skin surface have a diameter of about 10 μm. Due to confocal detection the source of collected Raman signals is well confined in all three dimensions inside a spot of a size smaller than 2×2×8 μm³. This allows collecting Raman signals from blood without background signal from skin tissue if the focus is located in a blood capillary. This spot location is possible if the lateral position of the blood vessel as well as the depth of the vessels below the skin surface are known with a resolution of 1 μm or better.

OPS imaging for blood vessel detection is also described in detail in European patent application 03100689.3. The analysis apparatus described therein produces a contrast image in a contrast wavelength range and a reference image in a reference wavelength range, said images being compared to accurately identify the target region, notably a capillary blood vessel in the patient's skin.

Because of an effective back-illumination of blood vessels, OPS imaging is essentially a 2-dimensional technique. The only depth information is obtained by the influence of the amount of (de)focus on the images. If an objective with a numerical aperture (NA) higher than 0.8 is used, the depth of field in skin is below 0.5 μm. Therefore, with accurate focusing algorithms based on image analysis it is possible to find the depth of the blood vessel.

Known auto-focusing methods are based on scanning the axial position of the objective focusing the imaging beam and the confocal excitation beam onto the object of interest, while measuring the value of a merit function to quantify the amount of (de)focus in the image. The best focus is found by optimizing the value of the merit function. In general there are many possibilities to change the focus position. For instance, one or two lenses in the objective can be moved (as in a photo camera) or the whole objective lens or another lens in the system can be moved. Also the shape of an optical element in the system can be changed, for example an electro wetting optical element. However, if the object is not known, the maximum of the merit function is also unknown. Therefore, the merit function provides only information about the amount of focus in relation to other focus positions.

Patients will move in lateral as well as in transversal directions. Therefore, continuous measurement and adjustment of the optimal location of the confocal detection center is required. Transversal movements in the image plane can easily be detected, whereas axial movements (perpendicular to the detection plane) are much more difficult to detect. A common method of detecting axial movement or defocus is by continuously moving the detection plane around the central best focus position (so called wobbling). This can be done by moving the imaging objective or another optical element in the imaging system. If the focus becomes better in front or behind the central position, the central position of the objective is changed. In known systems the detection volume is located in the image plane. Therefore this detection volume is also continuously moved around the best measurement position. This has the disadvantage that the confocal detection volume is located inside a blood vessel for only a fraction of time, and to avoid mixing of skin spectra with blood spectra, the intake of Raman signal has to be gated. This increases the time needed to collect sufficient Raman signal, which is in case of continuous recording already at least 30 sec.

Further disadvantages are, that due to changes in the blood flow the shape and size of a capillary change continuously; so that comparing images acquired at different times add uncertainty to the position of best focus. Additionally, the fact that more time is needed to collect sufficient Raman signal adds to the noise in the Raman spectrum because more dark current is acquired or because more readout noise is added.

It is therefore an object of the present invention to provide an optimized analysis apparatus and a corresponding analysis method for imaging and spectroscopic analysis of an object which allow continuous accurate auto-focusing of the excitation beam onto the object, in particular a blood vessel, even during movements of the object without changing the position of the detection volume continuously. Further, an optical focusing system for focusing on a target point of an object shall be provided, which system can, for instance, be applied in a tracking system for continuously tracking the target point in a moving object.

This object is achieved according to the present invention by an analysis apparatus as claimed in claim 1 comprising:

an excitation system for emitting an excitation beam to excite a target region,

a monitoring system comprising a monitoring beam source for emitting a monitoring beam and an imaging system to image the target region,

a detection system for detecting scattered radiation from the target region generated by the excitation beam,

focusing means for focusing the excitation system, the monitoring system and the detection system on a detection plane in the target region,

image processing means for determining image characteristics, which indicate if the imaging system is focused on the object to be analyzed, from a detected image, and

auto-focusing means for controlling the focusing means to change the focusing of the monitoring system, the excitation system and the detection system based on the determined image characteristics, for controlling the monitoring system to image the target region and for controlling the image processing means to determine the image characteristics from a detected image until the object substantially lies in the detection plane.

The object is further solved by a corresponding analysis method as claimed in claim 11. Preferred embodiments of the invention are defined in the dependent claims.

The invention is based on the idea to evaluate the detected image, to determine certain image parameters and to conclude therefrom if the imaging system, and thus also the excitation system and the detection system are focused on the object which shall be analyzed. The determined image characteristics are used to decide if the focusing needs to be changed or not. If the object does not yet lie in the detection plane, or in other words, if the focusing is not yet sufficient, the focusing is changed whereafter a new image is detected and new image characteristics are determined therefrom in order to again check if the focusing is sufficient This recursive procedure can be executed continuously during analysis of the object in order to ensure that the Raman confocal detection volume can be continuously located exactly inside the object of interest (such as a blood vessel).

Compared to other known auto-focusing techniques the present invention provides the advantage that the confocal detection volume is continuously located in the center of the object of interest, even if the object moves during the measurement. According to preferred embodiments no moving elements are required and a single microscope objective having a high numerical aperture can be used as focusing means. No continuous movement of the detection plane around the central best focus position (wobbling) is required. Another advantage of the present invention is that a simple, fast and robust (relative) focus measure is obtained which is required for many focusing methods, such as for continuous tracking methods or for just finding the right depth of a blood vessel a single time (e.g. before a Raman measurement to locate the depth of a capillary blood vessel).

Different image parameters are available which allow an indication if the imaging system is focused on the object of interest. According to a preferred embodiment as claimed in claim 2 the spatial frequencies corresponding to typical characteristics of the object of interest, e.g. to typical diameters of blood vessels during in vivo analysis of blood, are determined from a detected image. Since in focus the amplitudes of such spatial frequencies are maximally the focusing is changed until the determined amplitudes are maximally.

According to another preferred embodiment as claimed in claim 4 the maximum contrast present in a detected image and/or at one or more image portions corresponding to the object or object portions, e.g. the maximum contrast present in a detected image between blood and surrounding tissue, in particular at the edges of blood vessels, during in vivo analysis of blood, are determined. Since in focus the contrast is maximally, the focusing is changed until the determined contrast is maximally.

Preferred embodiments based on maximizing the contrast are defined in dependent claims 6 to 9.

It is preferred that the monitoring system is adapted for orthogonal polarized spectral imaging as mentioned above and as described in W002/057759 A1 and in European patent application 03100689.3.

The invention can not only be used in an analysis apparatus as described above, but relates also to an optical focusing system for focusing on a target point of an object, comprising a target system to be focused on the target point, a monitoring system, focusing means, image processing means and auto-focusing means as claimed in claim 12. The invention can be used in every system where an imaging system is used to locate and continuously track a target point, for example the focus of a laser beam or the detection volume of a spectroscopic system, continuously in 3 dimensions at a specific position in a moving target. Examples include: (biomedical) laser surgery, laser cutting, laser welding, laser shaving, photodynamic therapy, remote sensing, and target and tracking in military applications. Also the above described analysis apparatus could be regarded as including such an optical focusing system.

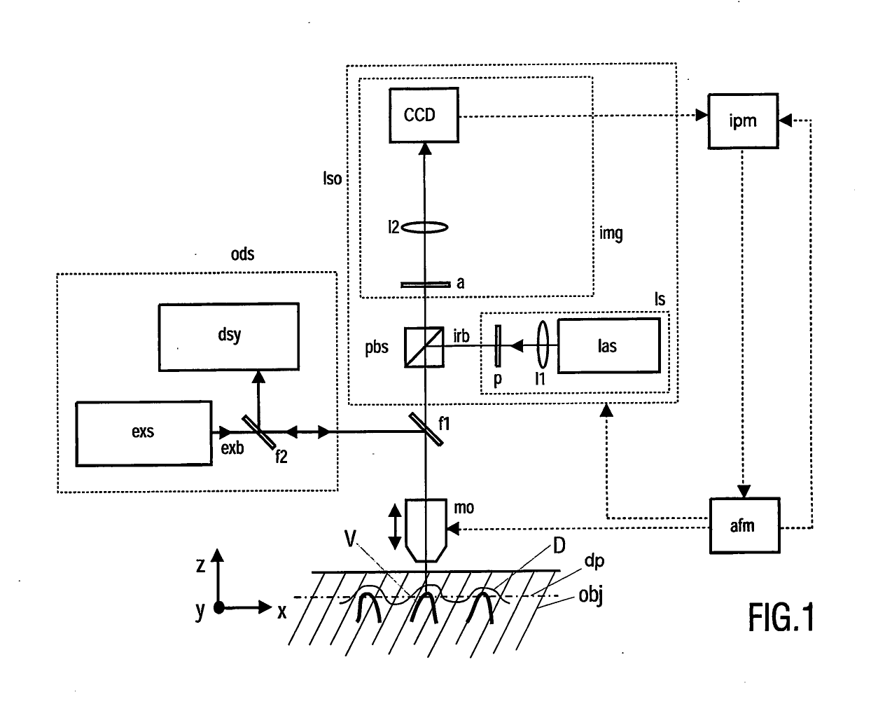

The invention will now be explained in more detail with reference to FIG. 1 which shows a graphic representation of an embodiment of an analysis system according to the present invention.

FIG. 1 is a graphic representation of a preferred embodiment of an analysis system in accordance with the invention. The analysis system includes an optical monitoring system (lso) for forming an optical image of the object (obj) to be examined. In the present example the object (obj) is a piece of skin of the forearm of the patient to be examined. The analysis system also includes a multi-photon, non-linear, elastic or inelastic scattering optical detection system (ods) for spectroscopic analysis of light generated in the object (obj) by a multi-photon, non-linear, elastic or inelastic optical process. The example shown in FIG. 1 utilizes in particular an inelastic Raman scattering detection system (dsy) in the form of a Raman spectroscopy device. The term optical encompasses not only visible light, but also ultraviolet radiation and infrared, especially near-infrared radiation.

The monitoring system (lso) comprises a monitoring beam source (ls) for emitting a monitoring beam (irb) and an imaging system (img) for imaging the target region, e.g. a blood vessel (V) in the upper dermis (D) of the patient's forearm (obj). The monitoring beam source (ls) in this example comprises a white light source (las), a lens (11) and an interference filter (not shown) to produce light in the wavelength region of 560-570 nm. Further, a polarizer (p) for polarizing the monitoring beam (irb) is provided. The monitoring beam source (ls) is thus adapted for orthogonal polarized spectral imaging (OPS imaging).

In OPS imaging polarized light is projected by a microscope objective (mo) through a polarizing beam splitter (pbs) onto the skin (obj). Part of the light reflects directly from the surface (specular reflection). Another part penetrates into the skin where it scatters one or more times before it is absorbed or is re-emitted from the skin surface (diffuse reflection). In any of these scattering events the polarization of the incident light is slightly changed. Light that is directly reflected or penetrates only slightly into the skin will scatter only one or a few times before it is re-emitted, and will mostly retain its initial polarization. On the other hand, light that penetrates more deeply into the skin undergoes multiple scattering events and is completely depolarized before re-emitted back towards the surface.

When looking at the object (obj) through a second polarizer or analyzer (a), oriented precisely orthogonal to that of the first polarizer (p), light reflected from the surface or the upper parts of the skin is largely suppressed, whereas light that has penetrated deep into the skin is mostly detected. As a result the image looks as if it were back-illuminated. Because wavelengths below 590 nm are strongly absorbed by blood, the blood vessels appear dark in the OPS image.

Generally, an image is obtained using a monochrome CCD camera. Blood vessels are separated from other absorbing structures be means of size, shape and movement of blood cells. The imaging system (img) used in the present embodiment comprises an analyzer (a) mentioned above for allowing only light having a polarization orthogonal to the light of the polarized monitoring beam (irb) to pass which is reflected back through the polarizing beam splitter (pbs) from the object (obj). Said light is further focused by a lens (12) onto the CCD-camera (CCD).

The Raman spectroscopy device (ods) comprises an excitation system (exs) for emitting an excitation beam (exb) and a detection system (dsy) for detection of Raman scattered signals from the target region. The excitation system (exs) can be constructed as a diode laser which produces the excitation beam in the form of an 785 nm infrared beam (exb). Of course other lasers can be used as excitation system as well. A system of mirrors and, for instance, an optical fiber conduct the excitation beam (exb) to a dichroic mirror (f1) for conducting the excitation beam (exb) along the monitoring beam (irb) to the microscope objective (mo) for focusing both beams onto the object (obj).

The dichroic mirror (f1) also separates the return (monitoring) beam from scattered Raman signals. While the reflected monitoring beam is transmitted to the imaging system (img), elastically and inelastically scattered Raman light from the object is reflected at the dichroic mirror (f1) and conducted back along the light path of the excitation beam. Inelastically scattered Raman light is then reflected by an appropriate filter (f2) and directed along the Raman detection path in the detection system (dsy) to the input of a spectrometer with a CCD detector. The spectrometer with the CCD detector is incorporated into the detector system (dsy) which records the Raman spectrum for a wavelength range of 800 to 1050 nm. The output signal of the spectrometer with the CCD detector represents the Raman spectrum of the Raman scattered infrared light The signal output of the CCD detector is connected to a spectrum display unit, for example a workstation that displays the recorded Raman spectrum on a monitor. Also a calculation unit (e.g. a workstation) is provided to analyze the Raman spectrum and calculate the concentration of one or more analytes.

Regarding further details of the analysis apparatus in general and the function thereof reference is made to the above mentioned WO 02/057759 A1 and to European patent application 03100689.3.

To achieve continuous auto-focusing of the confocal Raman system (ods) in a blood vessel (V), image processing means (ipm) and auto-focusing means (afm) are provided. Such auto-focusing is required to locate a blood vessel and to aim the Raman system at this blood vessel. Since patients will move during a blood analysis in lateral (z) as well as in transversal (x, y) directions, continuous determination and adjustment of the optimal location of the confocal detection center is required. Transversal movements can be easily detected by the imaging system, whereas axial movements are much more difficult to detect.

According to the present invention the image processing means (ipm) process an acquired image of the object (obj) and determine certain image characteristics which indicate if the imaging system (img), and thus also the excitation system (exs) and the detection system (dsy) are focused on the object (obj), in particular a blood vessel (V) or not, i.e. if the object of interest (the blood vessel) substantially lies in the detection plane (dp) on which the microscope objective (mo) is focused. Actually, only a relative focus measure can be determined if the object is not known, therefore, a focus measure is always compared to a focus measure at a different position. The position with the highest (or lowest) value of the focus measure is the position with best focus. Different image characteristics allow such an indication, preferably the amplitudes of spatial frequencies corresponding to typical diameters of blood vessels or the contsast between blood and surrounding tissue. Based on the determined image characteristics the auto-focusing means (afm) control the microscope objective (mo) to change the focusing thereof accordingly, i.e. to improve the focusing if possible. After this change the monitoring system (lso) is controlled to take another image of the object which is processed again by the image processing means (ipm) in the same way in order to check if the focusing has improved or not based on the same image characteristics, now determined in the new image. This iterative procedure is executed continuously or at predetermined time intervals during the whole blood analysis to compensate for movements of the patient during the analysis. Another use is to determine the position of best focus before the Raman measurement is started.

As mentioned above different image characteristics can be used. In the following preferred image characteristics which are used for automatic focusing in OPS imaging of blood vessels according to the present invention will be explained.

(1) A first preferred image characteristic is the typical dimensions of blood vessels. It is known that the blood capillaries near the skin surface have a typical diameter of 10 μm and a (visible) length of about 50 μm. The 2-D spatial Fourier transform can be used to find the proper focus by maximizing the amplitudes of spatial frequencies with wavelengths equal or shorter than these typical dimensions. This can be used for monochromatic as well as bichromatic OPS imaging, bichromatic OPS imaging meaning OPS imaging with a contrast wavelength and a reference wavelength as described in detail in European patent application 03100689.3.

(2) A second preferred image characteristic is the contrast between blood and surrounding tissue. Before discussing auto-focusing methods based on contrast, two general remarks have to be made. In bichromatic OPS imaging almost all structures visible after subtraction of the red and yellow/green image are blood vessels. Therefore any method based on maximizing the contrast of an image automatically selects blood vessels. However, in monochromatic OPS imaging, blood vessels as well as other structures near the skin surface are visible. Therefore, care has to be taken to focus only on blood vessels. Other structures can be suppressed by averaging over a number of pixels and/or using a high-pass spatial Fourier filter. In a monochromatic OPS imaging system the (preprocessed) image is used for image analysis and blood vessels appear as dark structures on a light background. In bichromatic OPS imaging the difference image (yellow/green minus red) is used for processing and blood vessels appear as light structures on a dark background.

Auto-focusing methods based on contrast can be divided into four categories. For all methods the average intensity of the image is kept constant
(a) Maximize or minimize the intensity of a single pixel as a function of depth. Light originating from outside the focal plane (detection plane) will be spread over several pixels in the image. By this spreading, the intensity of bright areas decreases, whereas the intensity of dark regions increases. For monochromatic OPS imaging, blood vessels are more dark compared to surrounding tissue, hence blood vessels are in-focus if an image containing the darkest pixel is obtained. For bichromatic OPS imaging, blood vessels are more bright compared to surrounding tissue; hence blood vessels are in-focus if an image containing the brightest pixel is obtained. Mathematically this can be expressed as maximizing (I_{i,j})ₘₐₓ(z) or minimizing (I_{i,j})ₘᵢₙ(z). Here (I_{i,j})ₘₐₓ(z) and (I_{i,j})ₘᵢₙ(z) represent the intensity of the pixel with maximum or minimum intensity when the imaging system is focused at a depth z below the skin surface.
(b) Maximize the spread in intensity of pixels as a function of depth. Because light originating from outside the focal plane will be spread over several pixels in the image, the spread in the intensity distribution will decrease if blood vessels are out of focus. The best focus can be obtained by maximizing

$${\sum }_{i , j} {( {I}_{i , j} ( z ) - \overline{I} ( z ) )}^{2} .$$

Here I_{i,j} (z) is the intensity measured at a pixel with coordinates i,j and \overline{I}(z) is the intensity averaged over all pixels when the imaging system is focused at a depth z below the skin surface.
(c) Maximize the average intensity difference between neighboring pixels as a function of depth. Because light originating from outside the focal plane will be spread over several pixels in the image, the average intensity difference between adjacent pixels will decrease if the vessels are outside the focal plane. The best focus can be obtained by maximizing

$${\sum }_{i , j} {( {I}_{i , j} ( z ) - {I}_{i + 1 , j} ( z ) )}^{2} + {( {I}_{i , j} ( z ) - {I}_{i , j + 1} ( z ) )}^{2} .$$
(d) Maximize absolute difference between neighboring pixels as a function of depth. Instead of looking at the average intensity difference between neighboring pixels in the whole image, the best focus can be found by looking for the image with the largest contrast between neighboring pixels. Mathematically this can be expressed as maximizing |I_{i,j}(z)−I_{i+1,j}(z)|+|I_{i,j}(z)−I_{i,j+1}(z)|.

Each of the methods described above has its own advantages and disadvantages. Method (2a) has the advantage that only a single pixel (belonging to a single blood vessel) is considered. By maximizing the contrast, automatically the center of the thickest blood vessel close to the skin surface is selected. This is also the position that can be used most conveniently for Raman spectroscopic analysis of blood. Method (2d) looks at the edge of a single blood vessel. Therefore at a single blood vessel is focused, however, the optimum Raman measurement position is located in the center of the vessel. Because only one or two pixels are used in methods (2a) and (2d), these techniques are sensitive to noise.

Methods (1), (2b) and (2c) use the whole image to find the best focus, and therefore are less sensitive to noise. However, they find the best focus of multiple blood vessels. If these vessels are not on the same depth, the focal plane can lie in between the vessels. This problem can be solved using a region of interest containing a single blood vessel for focusing.

The contrast difference is only detectable for structures small compared to the intensity spreading due to defocus. Therefore defocus can easily be detected near the edge of structures. This is done in methods (2c) and (2d). The intensity in the center of the image of an object only changes due to defocus that causes a spread larger than the image of the structure. Methods based on the maximum or minimum intensity (2a) are therefore less sensitive to defocus.

Another preferred embodiment will be a combination of auto-focusing algorithms.
1. Use bichromatic OPS imaging to detect only structures that are caused by absorption of blood
2. Find the best average focus for multiple blood vessels by maximizing the average intensity difference between neighboring pixels (method 2c).
3. Select region of interest containing the image of a (part of a) single blood vessel.
4. Find the best focus for this vessel by
a. maximizing the average intensity difference between neighboring pixels (method 2c) or
b. minimize the intensity of a single pixel.

The choice for a or b depends on how the absolute intensity and the intensity difference depend on the amount of (de)focus.

The complete image can be used for auto-focusing. However, different blood vessels or parts thereof lie at different depths below the skin surface. Therefore, it is more accurate to use a region of interest around the best Raman measurement position for auto-focusing. In a different application with higher quality images, an accuracy of 1% of the depth of focus can be achieved with the method according to the present invention. Thus, for automatic focusing of the Raman excitation beam, the acquired accuracy in the order of 1 μm can be obtained.

In the above a monochromatic OPS imaging embodiment is described having a white light source and a filter. Nevertheless, the invention can be applied in many different embodiments including other monochromatic, bichromatic or multichromatic OPS imaging embodiments.

Compared to known auto-focusing techniques, the method according to the present invention has the advantage that the confocal detection volume can be continuously located in the center of the blood vessel in which the blood shall be analyzed. The images that are evaluated are preferably measured continuously. Further, the invention can be used as a simple, fast and robust (relative) method to obtain focus measure which can be used in many focusing methods, e.g. for just finding the right depth of a blood vessel a single time.

1. An analysis apparatus, in particular a spectroscopic analysis apparatus, for analyzing an object comprising:
an excitation system for emitting an excitation beam to excite a target region,
a monitoring system comprising a monitoring beam source for emitting a monitoring beam and an imaging system to image the target region,
a detection system for detecting scattered radiation from the target region generated by the excitation beam,
focusing means for focusing the excitation system, the monitoring system and the detection system on a detection plane in the target region,
image processing means for determining image characteristics, which indicate if the imaging system is focused on the object to be analyzed, from a detected image, and
auto-focusing means for controlling the focusing means to change the focusing of the monitoring system, the excitation system and the detection system based on the determined image characteristics, for controlling the monitoring system to image the target region and for controlling the image processing means to determine the image characteristics from a detected image until the object substantially lies in the detection plane.

2. An analysis apparatus as claimed in claim 1, wherein said image processing means are adapted for determining the amplitudes of spatial frequencies corresponding to typical characteristics of the object from a detected image and wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the determined amplitudes of spatial frequencies are maximally.

3. An analysis apparatus as claimed in claim 2, wherein said analysis apparatus is adapted for in vivo analysis of blood and wherein said image processing means are adapted for determining the amplitudes of spatial frequencies corresponding to typical diameters of blood vessels from a detected image.

4. An analysis apparatus as claimed in claim 1, wherein said image processing means are adapted for determining the maximum contrast present in a detected image and/or at one or more image portions corresponding to the object or object portions and wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the determined contrast is maximally.

5. An analysis apparatus as claimed in claim 4, wherein said analysis apparatus is adapted for in vivo analysis of blood and wherein said image processing means are adapted for determining the maximum contrast present in a detected image between blood and surrounding tissue, in particular at the edges of blood vessels.

6. An analysis apparatus as claimed in claim 4, wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the determined the intensity of one or more pixels in the detected image show an extremum.

7. An analysis apparatus as claimed in claim 4, wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the spread in intensity of pixels in the detected image is maximally.

8. An analysis apparatus as claimed in claim 4, wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the average intensity difference between neighboring pixels in the detected image is maximally.

9. An analysis apparatus as claimed in claim 4, wherein said auto-focusing means are adapted for controlling the focusing means to change the focusing of the monitoring system, the excitation system and the detection system based on the determined image characteristics, for controlling the monitoring system to repeatedly image the target region and for controlling the image processing means to determine the image characteristics from a detected image until the absolute intensity difference between neighboring pixels in the detected image is maximally.

10. An analysis apparatus as claimed in claim 1, wherein the monitoring system is adapted for orthogonal polarized spectral imaging, in particular for bichromatic orthogonal polarized spectral imaging.

11. An analysis method, in particular a spectroscopic analysis method, for analyzing an object comprising the steps of:
emitting an excitation beam by an excitation system to excite a target region,
emitting a monitoring beam by a monitoring system to image the target region by an imaging system,
detecting scattered radiation from the target region generated by the excitation beam by a detection system,
focusing the excitation system, the monitoring system and the detection system on a detection plane in the target region by a focusing means,
determining image characteristics, which indicate if the imaging system is focused on the object to be analyzed, from a detected image, and
controlling the focusing means to change the focusing of the monitoring system, the excitation system and the detection system based on the determined image characteristics, controlling the monitoring system to image the target region, and controlling the image processing means to determine the image characteristics from a detected image until the object substantially lies in the detection plane.

12. An optical focusing system for focusing on a target point of an object, comprising:
a target system to be focused on the target point,
a monitoring system comprising a monitoring beam source for emitting a monitoring beam and an imaging system to image the target region,
focusing means for focusing the target system and the monitoring system on a detection plane in the target region,
image processing means for determining image characteristics, which indicate if the imaging system is focused on the object to be analyzed, from a detected image, and
auto-focusing means for controlling the focusing means to change the focusing of the monitoring system and the target system based on the determined image characteristics, for controlling the monitoring system to image the target region and for controlling the image processing means to determine the image characteristics from a detected image until the object substantially lies in the detection planed.

13. An optical tracking system as claimed in claim 12, wherein said target system comprises a light beam generation means for emitting a light beam, in particular a laser for emitting a laser beam, to be focused on the target point of the object.

14. An optical tracking system as claimed in claim 12, adapted for use in the field of laser surgery, laser cutting, laser welding, laser shaving, photodynamic therapy, radio therapy, remote sensing and target and tracking.
